(19) United States
(12) Patent Application Publication
(10) Pub. No.: US 2022/0323000 A1
(43) Pub. Date: Oct. 13, 2022

(54) A Construction Method for Automatic Sleep Staging and Use Thereof

(71) Applicant: Jiangsu Aidi SciTech Research Institute Co., Ltd., NANJING (CN)

(72) Inventor: NORDEN EH HUANG, NANJING (CN)

(73) Assignee: Jiangsu Aidi SciTech Research Institute Co., Ltd., NANJING (CN)

(21) Appl. No.: 17/618,502

(22) PCT Filed: Jan. 15, 2021
(86) PCT No.: PCT/CN2021/071979

§ 371 Date: Dec. 13, 2021

(30) Foreign Application Priority Data
Dec. 24, 2020 (CN) 202011548920.9

(51) Int. Cl. A61B 5/00; A61B 5/374

(52) U.S. Cl. CPC A61B 5/4815; A61B 5/4812; A61B 5/374; A61B 5/7246; A61B 5/7264; A61B 5/4809

(57) ABSTRACT

The present invention provides a construction method for automatic sleep staging and use thereof. The construction method for automatic sleep staging comprises: acquiring a plurality of sets of PSG signals and manual sleep information of PSG signals; pre-analyzing to decompose the original time series in the PSG signals into a set of pseudo-intrinsic mode functions (pseudo-IMFs); assembling the pseudo-IMFs to obtain m sets of time series; analyzing by multiscale entropy (MSE), to calculate the entropy values of the m sets of time series on n coarse-graining timescales, thus obtaining an entropy matrix with m*n elements; establishing a correlation coefficient matrix between the levels of consciousness and the elements in the entropy matrix, and finding the coarse-graining timescale and filtering timescale corresponding to the most significantly positively correlated element or the most significantly negatively correlated element in the correlation coefficient matrix; and calculating the entropy value on the coarse-graining timescale and filtering timescale corresponding to the most significantly positively correlated element or the most significantly negatively correlated element, and assessing the sleep state according to the entropy value.

### FIELD OF THE INVENTION

The present invention relates to the field of data processing, in particular to a construction method for automatic sleep staging and use thereof.

### BACKGROUND OF THE INVENTION

We spend almost one third of our life in sleep. Sleep is a critical part of our lives. Its quality influences not only our general health but also our economic productivity. Though its function is not totally clear, recent research found the critical role in the waste cleansing by the shrinking of glial cells and the increase of cerebrospinal fluid flushing in addition to the traditional view of memory consolidation. Studies also reveal that the sleep is not a uniform state; the switches from one state to another are highly nonlinear and nonstationary processes. Sleep disorders have serious consequences including reduced quality of life, co-morbidities, early mortality, etc., and inflict huge impacts on economic societal costs. These make sleep research of critical importance. Traditionally, normal sleep is divided into five stages: the rapid eye movement (REM) and the other four non-rapid eye movement (NREM) stages (1-4) according to the criteria of Rechtschaffen and Kales (R&K). Sleep states are estimated based on the characteristics of polysomnography (PSG) recordings including signals of electroencephalograph (EEG), electromyography (EMG), and electrooculography (EOG) and other parameters. In clinical practices, sleep technicians visually determine the sleep stage for each 30 second epoch. However, the manual scoring is a time-consuming process, and results may be inconsistent by different readers due to similarities between different stages. Besides, the assessment of the quality of sleep through the polysomnography is a labor intensive, time consuming and error prone process. In addition, with the increase in the number of patients for sleep monitoring, it is increasingly difficult for limited sleep analysts to meet the increasing demand for PSG analysis.

Therefore, there is an urgent need for an accurate and objective method to automatically stage the sleep stages and assess the sleep quality.

### SUMMARY OF THE INVENTION

In order to solve the above problems, in the present invention, we propose intrinsic multiscale entropy (iMSE) as a new signal analysis method. First, we study sleep states with multiscale entropy (MSE), aiming to quantify complexity using the summation of entropies over multiple temporal timescales. In MSE, entropy is defined as a physical measure of ‘unpredictivity’ for a coarse-grained nonlinear time series on multiple timescales, which can be calculated by using the definitions of sample entropy or approximate entropy. The number of non-overlapping samples merged into one is defined as the timescale of coarse-graining in MSE. In the practices of digital signal processing, the timescale 1 represents the original time series measured and digitalized on the original sampling rate. Timescale n represents the coarse-grained time series with sampling interval n times of the original. The sampling rate is one nth of the original. Therefore, the timescale of coarse-graining represents the time length in number of the original sampling intervals. The approach by MSE reflects a view point that entropy is a measure which depends on the timescale of sampling interval. Second, as the sleep process is not stationary, we propose the intrinsic MSE (iMSE), which combines Empirical Mode Decomposition (EMD) with the MSE to resolve the non-stationary limitation. EMD enable us in two preprocessing steps of denoising and detrending in order to extract the desired information from nonlinear and nonstationary real-world signals. EMD acts as an adaptive dyadic filter bank to decompose a complicated time series into a set of intrinsic mode functions (IMFs). Each IMF is narrow band and zero-mean; therefore, stationary. The filtered time series can be reconstructed using different combinations of IMFs, in which high-frequency noisy component and/or low-frequency trends are excluded from the original time series. As a functional combination, we propose iMSE method as an innovation signal analysis method.

Furthermore, to avoid high computational cost and other subtle difficulties of EMD, we introduce a simple filter based pseudo-EMD method, mimicking the function of EMD to avoid the problem of mode mixing works to systemically extract filtered components from time series. Then, iMSE works to quantify the entropies of filtered components on multiple coarse-graining timescales (i.e., the sampling timescales). Meanwhile, the filtering frequency band represents the second timescale of filtering in iMSE. Then, the values of entropy are shown in a two-dimension matrix over two axes of coarse-graining timescale and filtering timescale that greatly enhance the original MSE capability.

In the present invention, we provide a method for automatic sleep staging, in which by analyzing the entropy matrix on the two axes of the coarse-graining timescale and the filtering timescale, the optimal coarse-graining timescale and filtering timescale suitable for automatic sleep staging are found. When analyzing a subject's sleep state, using this method only needs to calculate the entropy value on the optimal coarse-graining timescale and filtering timescale, that is, automatic sleep staging can be performed through the entropy value. This method will greatly reduce the amount of calculation of sleep staging with MSE, and further improve the speed of automatic sleep staging.

In order to achieve the above-mentioned purposes of the invention, the present invention provides a construction method for automatic sleep staging, which comprises the following steps: acquiring a plurality of sets of PSG signals and manual sleep information of PSG signals; pre-analyzing to decompose the original time series of each stage in the PSG signals into a set of intrinsic mode functions (IMFs) or pseudo-intrinsic mode functions (pseudo-IMFs); assembling the IMFs or pseudo-IMFs to obtain in sets of time series; analyzing by multiscale entropy (MSE), to calculate the entropy values of the m sets of time series on n coarse-graining timescales, thus obtaining an entropy matrix with m*n elements; establishing a correlation coefficient matrix between the level of consciousness and the elements in the entropy matrix, and finding the coarse-graining timescale and filtering timescale corresponding to the most significantly positively correlated element or the most significantly negatively correlated element in the correlation coefficient matrix, wherein the filtering timescale is a time series set; and calculating the entropy value on the coarse-graining timescale and filtering timescale corresponding to the most significantly positively correlated element or the most significantly negatively correlated element, and assessing the sleep state according to the entropy value.

Preferably, the original time series of each stage in the PSG signals is decomposed into a set of IMFs using the mode decomposition method, and the mode decomposition method is one of the following methods: an empirical mode decomposition method, an ensemble empirical mode decomposition method, and a conjugate adaptive dyadic masking empirical mode decomposition method.

Preferably, the original time series of each stage in the PSG signals is decomposed into a set of pseudo-IMFs using a set of high-pass filters, and the cut-off frequencies of the high-pass filters are 32 Hz, 16 Hz, 8 Hz, 4 Hz, 2 Hz, and 1 Hz, respectively.

Preferably, the PSG signals comprise at least one of the following Electroencephalogram (EEG) signals: Fp4-A1, F4-A1, C4-A1, P4-A1, and O2-A1.

Preferably, the level of consciousness is defined according to the manual sleep information, and the level of consciousness is used to reflect the degree of wakefulness during sleep, wherein a wake stage is quantified as 6, a rapid eye movement (REM) stage is quantified as 5, a non-rapid eye movement 1 (NREM1) stage is quantified as 4, an NREM2 stage is quantified as 3, an NREM3 stage is quantified as 2, and an NREM4 stage is quantified as 1.

Preferably, the correlation coefficient matrix between the level of consciousness and the elements in the entropy matrix is established based on Pearson coefficient.

Preferably, when determining the sleep state according to the entropy value on the coarse-graining timescale and filtering timescale corresponding to the most significantly positively correlated element or the most significantly negatively correlated element, the threshold between different sleep states is calculated using the artificial intelligence (AI) method.

The present invention also provides a method for automatic sleep staging, wherein it comprises the following steps: acquiring PSG signals of a subject; decomposing the PSG signals of the subject into original time series of a plurality of stages; decomposing the original time series of a stage into a set of IMFs or pseudo-IMFs; calculating the entropy value on the coarse-graining timescale and filtering timescale corresponding to the most significantly positively correlated element or the most significantly negatively correlated element; and assessing the sleep state of the subject at the stage according to the entropy value.

Preferably, the PSG signals comprise at least one of the following Electroencephalogram (EEG) signals: Fp4-A1, F4-A1, C4-A1, P4-A1, and O2-A1.

Preferably, when decomposing the PSG signals of the subject into original time series of a plurality of stages, the time of each stage is 30 seconds.

Through the present invention, we can construct an automatic sleep staging method, which only needs to measure the entropy value of a subject on the optimal coarse-graining timescale and filtering timescale, that is, automatic sleep staging can be performed through the entropy value. This method will greatly reduce the amount of calculation of sleep staging with MSE, and further improve the speed of automatic sleep staging.

### BRIEF DESCRIPTION OF THE DRAWINGS

FIG. 1 is a flow chart of the construct method for automatic sleep staging of the present invention.

FIG. 2 is five-channel EEG images for six different sleep stages.

FIG. 3 shows a typical set of pseudo-IMFs for six sleep stages (the channel is the C4-A1.).

FIG. 4 is a flow chart of the method for decomposing and re-assembling the original time series to obtain the time series set in the present invention.

FIG. 5 is the calculation of the entropy values on coarse-graining timescales from 1 to n for each set of time series.

FIG. 6 is an example diagram of two-dimensional entropy matrixes for six different sleep stages.

FIG. 7 is the correlation coefficient matrixes between the DCL and the individual entropy matrix elements in five entropy matrixes for five channels of EEG recordings.

FIG. 8a is the time series and trends of the manual sleep stages, PEDCL, and NEDCL, for channel of F4-A1; FIG. 8b is the time series and trends of the manual sleep stages, PEDCL, and NEDCL, for channel of C4-A1.

FIG. 9a is the results of an intra-subject comparison of the PEDCL values for the six sleep states of five channels; FIG. 9b is the result of an intra-subject comparison of the NEDCL values for the six sleep states of five channels.

FIG. 10a is the results of an inter-subject comparison of the PEDCL values for the six sleep states of five channels; FIG. 10b is the results of an inter-subject comparison of the NEDCL values for the six sleep states of five channels.

FIG. 11 is a flow chart of the method for automatic sleep staging of the present invention.

### DETAILED DESCRIPTION OF THE INVENTION

In the following, with the accompanying drawings and preferred embodiments of the present invention, the technical means adopted by the present invention to achieve the intended purposes of the invention are further described.

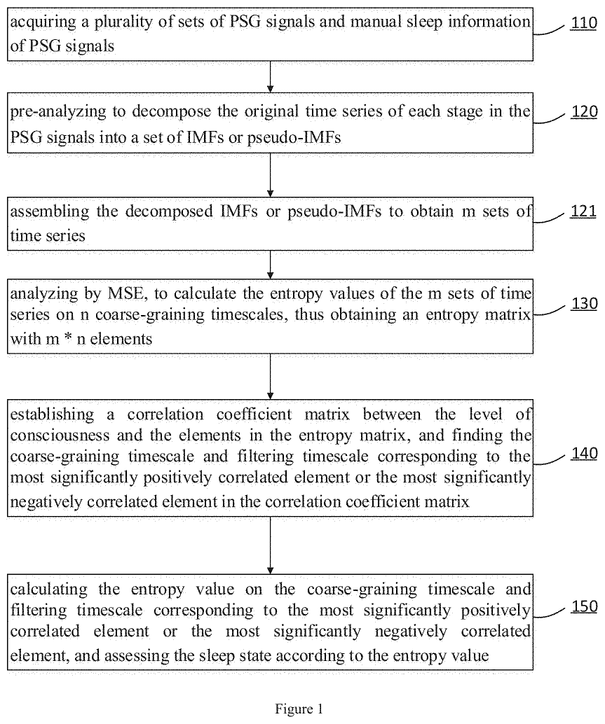

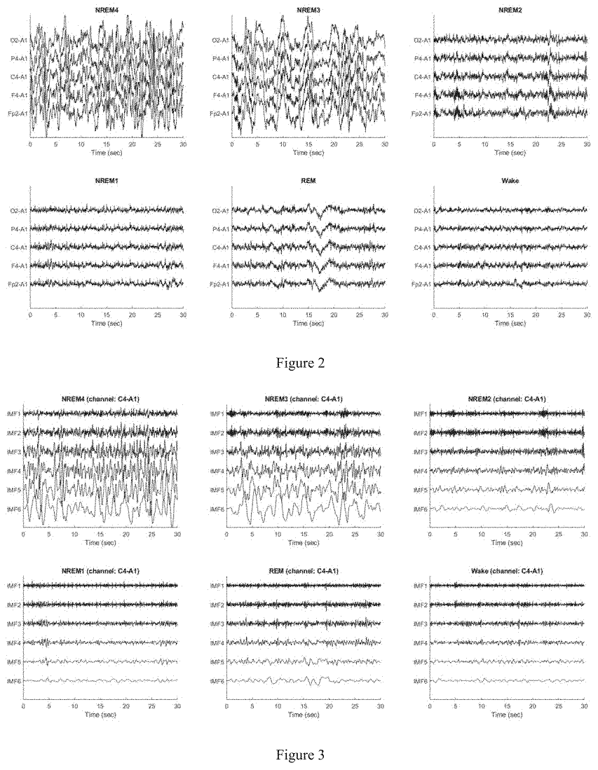

FIG. 1 is a detailed implementation of the method for automatic sleep staging of the present invention. Step 110 is acquiring a plurality of sets of PSG signals and manual sleep information of PSG signals. The polysomnography instrument can be used to measure and record one or more physiological signals, such as F3-A2 brain wave signal, F4-A1 brain wave signal, C3-A2 brain wave signal, C4-A1 brain wave signal, P3-A2 brain wave signal, P4-A1 brain wave signal, left eye movement signal, right eye movement signal, etc. Since sleep is a brain wave activity, the closer the physiological signal to the brain is, the more it can reflect the sleep state. Generally, brain wave signals are used for sleep staging. The cyclic alternating pattern (CAP) in sleep is a cyclical brain wave change that occurs during non-rapid eye movement sleep. This component reflects the microstructure of sleep. The polysomnography (PSG) recordings in the cyclic alternating pattern (CAP) sleep database can be download from website of PhsioNet. This database in PhsioNet provides a total of 108 PSG recordings with eight different pathological conditions. In the present invention, only three conditions of normal control, insomnia, and narcolepsy were selected for research. In order to reduce the variables of the automatic sleep staging method of the present invention, we also require that the electroencephalogram (EEG) channels and sampling frequencies recorded in these polysomnogram recordings must be consistent. Based on these criteria, we picked up the PSG recordings with five EEG channels of Fp4, F4, C4, P4, and O2 in sampling rate of 512 Hz. A subset of six normal control subjects (n1, n2, n3, n5, n10, and n11), five insomnia subjects (ins2, ins4, ins6, ins7, and ins8), and five narcolepsy subjects (narco1, narco2, narco3, narco4, and narco5) were used in this study. For each PSG recording, clear notations of event times, sleep stages and the CAP annotations were included in the text files. FIG. 2 is a typical five-channel EEG images of sleep states. The sleep states include a wake stage, a non-rapid eye movement stage (including the first to fourth stages, respectively denoted as NREM1, NREM2, NREM3 and NREM4) and a rapid eye movement (REM) stage. FIG. 2 shows six examples with five channels of EEG for six different sleep stages respectively. It can be seen from FIG. 2 that the EEG signals of NREM3 and NREM4, which represent deep sleep stages, contain low-frequency oscillations. Therefore, NREM3 and NREM4 are also noted as slow-wave sleep stages.

Step 120, decompose the original time series of each stage in the PSG signals into a set of IMFs or pseudo-IMFs. In the PSG recordings, sleep is divided into 30 seconds per stage, and sleep states are then analyzed. Therefore, when we establish an automatic sleep staging method in the present invention, we also analyze a stage of 30 seconds, and decompose the original time series of each stage in the PSG signals into a set of IMFs or pseudo-IMFs. The essence of the pre-analysis is to decompose the original time series into a set of independent narrow bands and detrended zero-mean IMFs or pseudo-IMFs with dyadic frequency bands.

This step is essential, for the entropy is computed from probability density function of the data. But the probability density can only be performed on data with no trend. When decomposing the original time series, the mode decomposition method can be used. EMD is an ideal dyadic filter bank to adaptively decompose a nonlinear time series into a set of IMFs. The mode decomposition method refers to any mode decomposition method that can obtain the IMF components in the present invention, such as empirical mode decomposition (EMD), ensemble empirical mode decomposition (EEMD) or conjugate adaptive dyadic masking empirical mode decomposition (CADM-EMD).

In the present invention, we also provide an alternative method to overcome the shortcomings of mode decomposition method for sleep staging. The mode decomposition method used for sleep staging has the following shortcomings: Firstly, the mode-mixing problem is hard to be totally resolved unless expensive computational and carefully designed masking methods are used in the enhanced algorithms. Otherwise, the resulting IMFs might have mis-matched intrinsic frequency bands of different IMFs in different sleep stages. For example, the distribution of instantaneous frequency of IMF 1 from an EEG signal recorded on a sleep stage of NREM1 is different from that of EEG signal recorded on a sleep stage of NREM4. Secondly, it is not easy to align the resulting frequency bands from the IMFs with the commonly defined bands for EEG signals. For example, the commonly defined delta band is for 0.5 to 4 Hz, theta band for 4 to 8 Hz, alpha band for 8 to 16 Hz, beta band for 16 to 30 Hz, and gamma band for 30 to 60 Hz. These frequency bands are similar but not exactly the same. To facilitate comparisons with the commonly defined frequency bands, we can simulate a predetermined (but not adaptive) filter bank to extract a set of pseudo-IMF functions from an EEG signal in an alternative method of EMD. And thirdly, for those unfamiliar with EMD, the filter method is easier to implement. In the alternative method of the present invention, a low-pass filter with cutoff frequency of 64 Hz is used to remove the high-frequency noise from the time series firstly. Then, a set of high-order high-pass filters with cutoff frequencies of 32, 16, 8, 4, 2, 1 Hz in sequence are used to extract the first six IMFs. Theoretically, the frequency bands of first six IMFs decomposed by the alternative method are 32-64 Hz (similar to gamma band), 16-32 Hz (beta band), 8-16 Hz (alpha band), 4-8 Hz (theta band), 2-4 Hz (delta band), and 1-2 Hz (low delta band), respectively, as shown in Table 1.

TABLE 1

| Filtered component | Frequency range | Frequency band |
|---|---|---|
| pseudo-IMF1 | 32-64 Hz | Gamma band (γ) |
| pseudo-IMF2 | 16-32 Hz | Beta band (β) |
| pseudo-IMF3 | 8-16 Hz | Alpha band (α) |
| pseudo-IMF4 | 4-8 Hz | Theta band (θ) |
| pseudo-IMF5 | 2-4 Hz | Delta band (δ) |
| pseudo-IMF6 | 1-2 Hz | Low delta band (Lδ) |

By using the pseudo-IMFs obtained by the filter method, the problem that the frequency bands from the IMFs are not easy to align with the general EEG frequency bands can be solved well. FIG. 3 shows a typical set of pseudo-IMFs obtained by the method for six sleep stages. The EEG channel selected in the figure is the C4-A1. The filtering bands of pseudo-IMFs 1-6 are γ band (32-64 Hz), β band (16-32 Hz), α band (8-16 Hz), θ band (4-8 Hz), δ band (2-4 Hz), and low δ band (1-2 Hz) respectively.

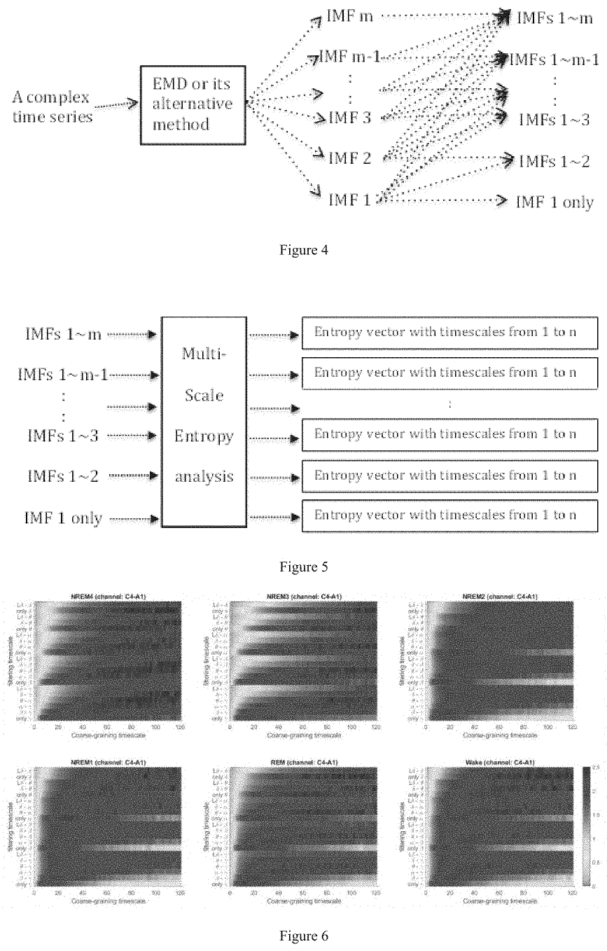

Step 121 is assembling the decomposed IMFs or pseudo-IMFs to obtain m sets of time series. These sets of filtered time series can be reconstructed using the various assembles of the IMFs or pseudo-IMFs into a new assembly of m-set of detrended zero-mean time series, which present the original data from different perspective points, such as only the high frequency components, or any specific selected frequency bands. As shown in FIG. 4, the process of step 120 to step 121 is shown. Decompose a set of complex time series by mode decomposition or its alternative method to obtain a set of IMFs or pseudo-IMFs, and then assemble the set of IMFs or pseudo-IMFs. The reconstructed filtered time series will cover all possible frequency bands of the original data. Taking the above six pseudo-IMFs as an example, from these first 6 pseudo-IMFs, 14 additional filtered time series can be reconstructed for further analysis. The 20 filtered time series include only IMF 1, IMFs 1-2, IMFs 1-3, IMFs 1-4, IMFs 1-5, IMFs 1-6; only IMF 2, IMFs 2-3, IMFs 2-4, IMFs 2-5, IMFs 2-6; only IMF 3, IMFs 3-4, IMFs 3-5, IMFs 3-6; only IMF 4, IMFs 4-5, IMFs 4-6; and only IMF 5, IMFs 5-6.

Step 130 is analyzing by multiscale entropy (MSE), to calculate the entropy values of the m sets of time series obtained in step 121 on n coarse-graining timescales respectively, thus obtaining an entropy matrix with m*n elements. As shown in FIG. 5, for each set of time series, the entropy values on coarse-graining timescales from 1 to n were calculated to obtain an entropy matrix with m*n elements. MSE evaluates the complexity of time series based on entropy values corresponding to multiple different timescales. In iMSE, the entropy value for each re-assembled filtered time series, which contains one or more IMFs or pseudo-IMFs, can be estimated using multiple coarse-graining timescales to obtain a row vector of entropy. Coarse-graining timescale is defined as the number of successive samples from the original time series non-overlappingly merged into a sample in the coarse-grained time series. The length of a coarse-grained time series is one n-th of the length of the original time series, wherein n is the coarse-graining timescale. Only the time series with coarse-graining timescale of 1 is the original time series. One entropy value can be calculated for coarse-grained time series using any definition of entropy, such as approximate entropy, sample entropy, etc. In the present invention, approximate entropy (ApEn) is selected to calculate the entropy vector for a filtered time series in MSE. In order to reduce the impact of individual cases and different sleep stages on the entropy values, and objectively display the entropy values of each different sleep state, multiple sleep stages can be selected for research. According to step 120 to step 130, respectively, the entropy matrixes of each sleep stage are obtained, and then the average value of the entropy matrixes with the same manual scored sleep stages is calculated to obtain the average entropy matrix. As shown in FIG. 6, it is the results of calculating the entropy values of the time series on 20 different filtering timescales in the present invention from 60 coarse-graining timescales from 2 to 120 with step of 2. A two-dimension average entropy matrix with 20*60 elements were estimated for 20 filtered time series on 60 different coarse-graining timescales. FIG. 6 represents six typical average entropy matrixes for six different sleep stages. In these sub-figures, entropy values are shown in colors, the X-axis notes the coarse-graining timescales from 2 to 120 with step of 2, and the Y-axis notes the filtering timescales in pseudo-IMFs from 1 to 20. Each entropy matrix represents the entropy measure for EEG signals of an epoch on multiple control conditions of filtering and sampling. There are obvious differences among the different sleep stages.

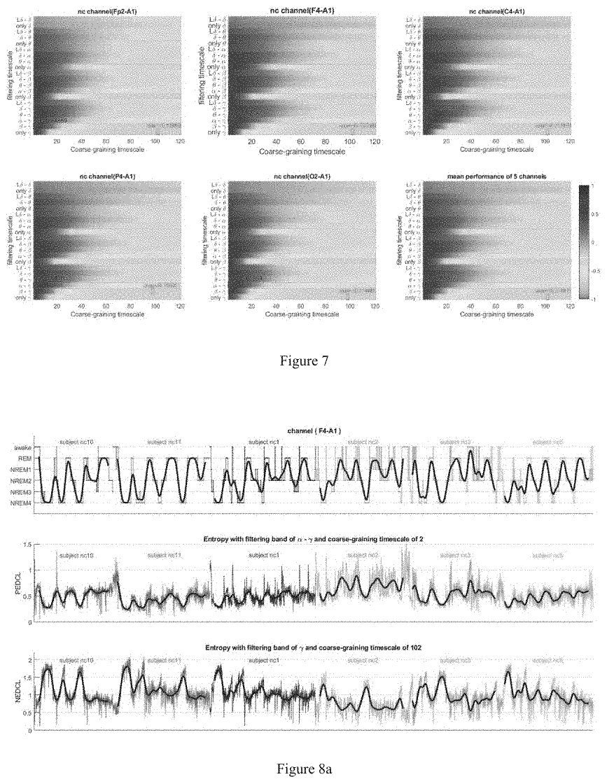

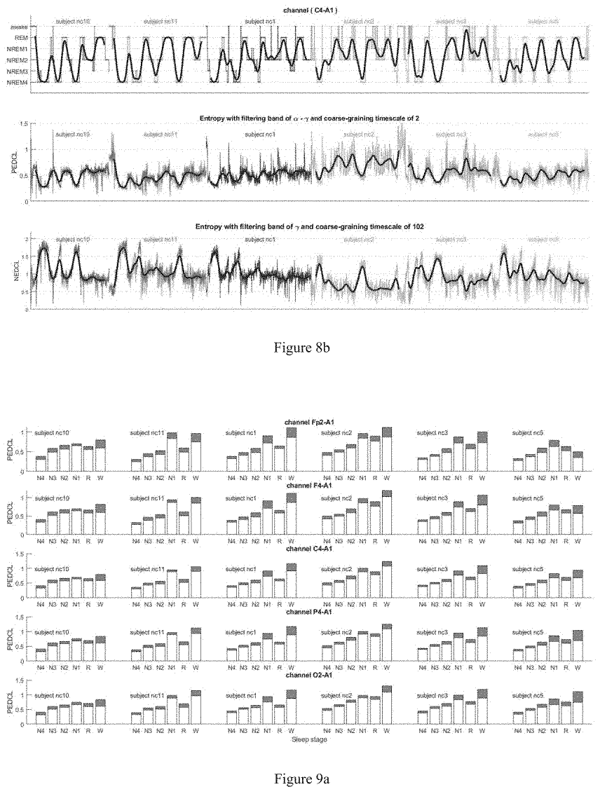

Step 140 is establishing a correlation coefficient matrix between the level of consciousness and the elements in the entropy matrix, and finding the coarse-graining timescale and filtering timescale corresponding to the most significantly positively correlated element or the most significantly negatively correlated element in the correlation coefficient matrix. The level of consciousness is defined according to the manual sleep information of PSG signals. In order to establish the relationship between the level of consciousness and the entropy value, firstly define the discrete consciousness level (DCL) in sleep according to the manual scored sleep stages, and the level of consciousness is used to reflect the degree of wakefulness during sleep. A wake stage representing the highest level of consciousness is quantified as 6, a rapid eye movement (REM) stage is quantified as 5, an NREM1 stage is quantified as 4, an NREM2 stage is quantified as 3, an NREM3 stage is quantified as 2, and an NREM4 stage representing the lowest level of consciousness is quantified as 1. On average for each subject, there will be nearly 1000 epochs over a night of sleep. This formed a time series with the above defined magnitude designated as DCL series. Next, correlation between DCL series and the time series of each iMSE matrix element was examined. We found that some elements are positively correlated to the DCL in sleep, and some of the others are negatively correlated based on Pearson correlation coefficient. The correlation coefficient matrixes between the DCL and the individual elements in five entropy matrixes for five channels of EEG recordings are shown in FIG. 7. The first five sub-figures represent the correlation coefficient matrixes derived from five different EEG channels, and the sixth sub-figure shows the average matrix of the first five. Then, we pick up one element with the most significantly positively correlated to DCL series and one with the most significantly negatively correlated to DCL series from each correlation coefficient matrix. For example, in the matrix for channel Fp2-A1 (sub FIG. 1), the most significantly positively correlated element, with correlation coefficient of 0.64869, has filtering timescale of IMFs 1-3 (α-γ band) and coarse-graining timescale of 2 (coarse-graining timescale of 2/512 seconds in sampling rate of 512 Hz); while the most significantly negatively correlated element, with correlation coefficient of −0.73802, has filtering timescale of IMF 1 only (γ band) and coarse-graining timescale of 102 (coarse-graining timescale of 102/512 seconds in sampling rate of 512 Hz). This location is common to all six subjects and over all five electrodes. We can select the coarse-graining timescale and filtering timescale as the optimal coarse-graining timescale and filtering timescale of the subject, and then automatically sleep stage the subject according to the entropy value on the selected coarse-graining timescale and filtering timescale. This is true as indicated in the average matrix in the last figure. Thus, we decide to pick these two entropy elements according to the ensemble result as shown in sub-FIG. 6 to represent all the subjects. In the present invention, in addition to using the entropy values on the coarse-graining timescale and filtering timescale position of the most significantly positively correlated element or the most significantly negatively correlated element, one can also select the entropy values of several coarse-graining scale and filtering timescale positions near the most significantly positively correlated element or the most significantly negatively correlated element to increase the adaptability of this automatic sleep staging method. As shown in FIG. 8, we studied the positive and negative relationship between intrinsic entropy and discrete consciousness level. The entropy element positively correlated to DCL series is noted as positive entropy for DCL (PEDCL), and the entropy element negatively correlated to DCL series is noted as negative entropy for DCL (NEDCL). FIG. 8 shows the manual sleep stages, PEDCL, and NEDCL, for two EEG channels of F4-A1 (FIG. 8a) and C4-A1 (FIG. 8b). All PEDCL and NEDCL values for all the epochs together with the trend (actual individual epoch readings filtered by a digital lowpass filter with cut-off frequency of 1 cycle per hour) are shown. The sampling rates of manual sleep stages, PEDCL, and NEDCL, are 120 cycles per hour in sleep staging using 30-seconds epochs. As shown in FIG. 8, PEDCL and the manual sleep stage trends are similar; while NEDCL and the manual sleep stage trends are inversely related. Both results derived from channels of F4-A1 and C4-A1 perfectly match the manual sleep cycles for six different subjects. Therefore, the automatic sleep staging method provided in the present invention will be able to stage the sleep states well.

Step 150 is calculating the entropy value on the coarse-graining timescale and filtering timescale corresponding to the most significantly positively correlated element or the most significantly negatively correlated element, and determining the sleep state according to the entropy value. In the present invention, one can also select the entropy values of several sampling scale and filtering timescale positions near the most significantly positively correlated element or the most significantly negatively correlated element to increase the adaptability of this automatic sleep staging method.

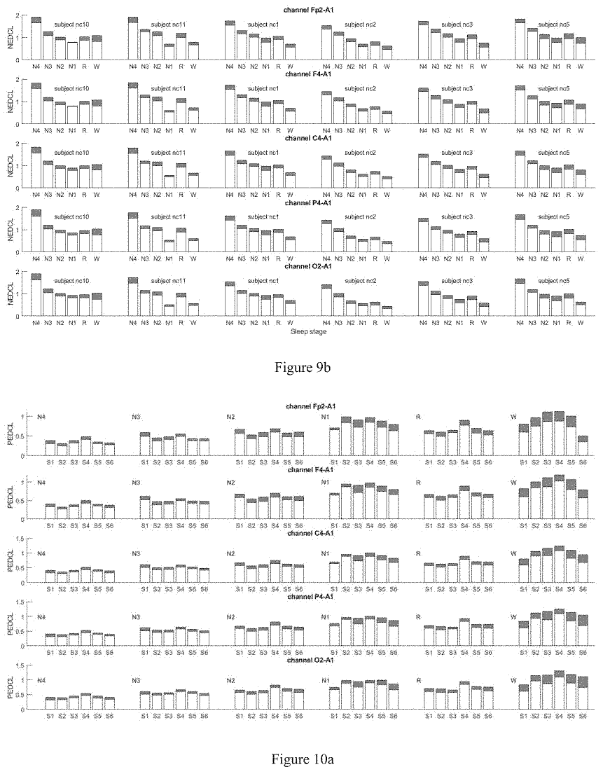

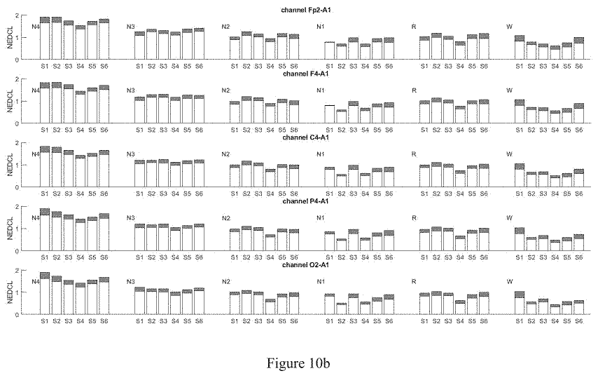

In order to verify whether the value of the discrete consciousness level is reasonable, we studied whether the complexity measure carried out in the intrinsic multi-scale entropy is consistent with the value of the discrete consciousness level. It should be pointed out that the aforementioned discrete level of consciousness (DCL) with values from 1 to 6 according to the manual sleep stages is not a linear scale. However, it is certainly true that the consciousness level of NREM3 is theoretically higher than that of NREM4, but there is no linear relationship between the consciousness levels of NREM4 and NREM3. It is logic to define the consciousness of wake stage as the highest level in sleep cycle, and the consciousness levels for four NREM sleep stages should be in a sequence of NREM1>NREM2>NREM3>NREM4. Therefore, PEDCL is defined as a practical entropy measure with positive correlation to the consciousness level in sleep. Now, it is critical to verify whether the complexity measure via iMSE agrees with this consciousness designation. The results of an intra-subject statistical comparison for six sleep stages are given in FIG. 9, in which the values of the means and standard deviations of PEDCL are presented for all subjects and all of five EEG channels. FIG. 9a is the results of an intra-subject comparison of the PEDCL values for the six sleep states of five channels; FIG. 9b is the result of an intra-subject comparison of the NEDCL values for the six sleep states of five channels. In general, the results support the ranking of PEDCL values in a sequence of wake>NREM1>NREM2>NREM3>NREM4. In the figures, the NREM4 is abbreviated as ‘N4’; NREM3 as ‘N3’; NREM2 as ‘N2’; NREM1 as ‘N1’; REM as ‘R’; and wake as ‘W’. In contrast to the results of PEDCL, NEDCL value is in a sequence of wake<NREM1<NREM2<NREM3<NREM4. The only exception is for REM. Although REM sleep stage was discovered more than 50 years ago, the neuronal circuit switch between REM and Non-REM sleep is still poorly understood. Consequently, the REM stage is known as paradoxical sleep. There have been studies that have proposed a brain stem flip-flop control of the switch between REM and non-REM sleep stages. Importantly, our results indicate that the PEDCL and NEDCL values for REM are closer to those for the NREM2 stage: For PEDCL, value of REM stage is lower than both NREM1 and wake stage; for NEDCL, value of REM stage is higher than both NREM1 and wake stage. However, this unique characteristic is enough for us to classify the sleep stage based solely on EEG recordings. Coupled with electrooculography (EOG) and muscle tone data, we can remove any ambiguity and make this classification definitive easily. Next, we will examine the inter-subject comparisons for different sleep stages. The results are given in FIG. 10, in which the mean and standard deviations for inter-subject comparisons among six subjects for both PEDCL and NEDCL values are given. FIG. 10a is the results of an inter-subject comparison of the PEDCL values for the six sleep states of five channels; FIG. 10b is the results of an inter-subject comparison of the NEDCL values for the six sleep states of five channels. None of the values for individual subject differs from the mean significantly (with statistical significance tested by Kolmogorov-Smimov, p<0.05). These results indicate that PEDCL and NEDCL can be used as objective quantitative measures for reflecting the fluctuation patterns of sleep cycles based on the dynamic ranges of PEDCL and NEDCL. For the purpose of automatic sleep staging, the dynamic range should be considered to determine the thresholds for classifications among sleep stages.

Through the present invention, we can establish an automatic sleep staging method, which only needs to measure the entropy value of a subject on the optimal coarse-graining timescale and filtering timescale, that is, automatic sleep staging can be performed through the entropy value. This method will greatly reduce the amount of calculation of sleep staging with MSE, and further improve the speed of automatic sleep staging.

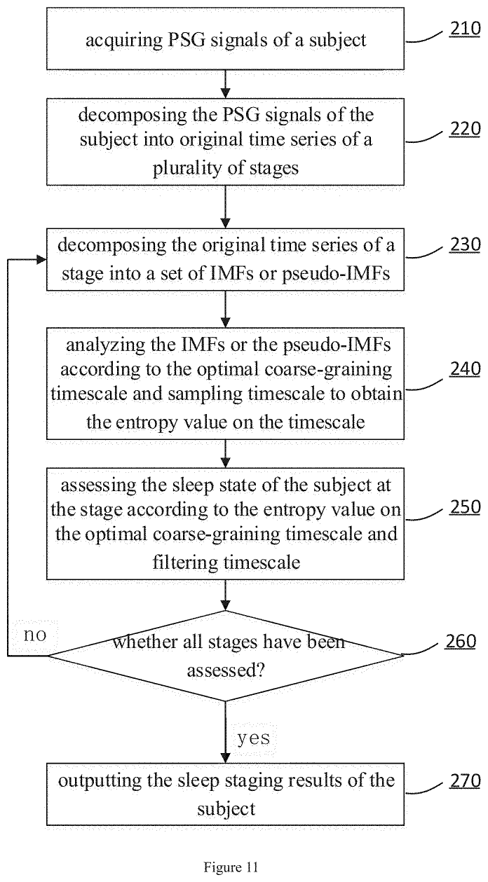

As shown in FIG. 11, the present invention also provides a method for automatic sleep staging. The method for automatic sleep staging comprises: step 210, acquiring PSG signals of a subject, and staging sleep using the PSG signals. Step 220, decomposing the PSG signals of the subject into original time series of a plurality of stages. The time of each stage can be consistent with the method for automatic sleep staging. The current manual staging uses 30 seconds as a stage. The present invention is not limited to this, and the time of each stage can also be shortened to better check the influence of the sleep state on other aspects of health. Step 230, decomposing the original time series of a stage into a set of IMFs or pseudo-IMFs. In this step, the decomposition method of the original time series is the same as step 120 in the automatic sleep staging method. Step 240, analyzing the IMFs or the pseudo-IMFs according to the optimal filtering timescale and coarse-graining timescale to obtain the entropy value on the timescale. Here, the optimal filtering timescale and coarse-graining timescale refer to the coarse-graining timescale and filtering timescale of the most significantly positively correlated element or the most significantly negatively correlated element in the correlation coefficient matrix in the automatic sleep staging method. In FIG. 8, it can be seen that the entropy values on the optimal filtering timescale and coarse-graining timescale are consistent with the results of manual sleep staging, and the entropy values on the timescale are very suitable for manual sleep staging. Step 250, assessing the sleep state of the subject at the stage according to the entropy value on the optimal coarse-graining timescale and filtering timescale. Step 260, determining whether all stages have been assessed. If it is not completed, return to step 230 to obtain the original time series of the next stage; if all the stages are assessed, output the sleep staging results of the subject. With the automatic sleep staging method of the present invention, it is only necessary to calculate the entropy value of a subject on the optimal coarse-graining timescale and filtering timescale, that is, automatic sleep staging can be performed through the entropy value. This method will greatly reduce the amount of calculation of sleep staging with MSE, and further improve the speed of automatic sleep staging.

In order to overcome individual differences and different threshold values between different sleep states, the present invention also provides an artificial intelligence method for assisting sleep staging. This artificial intelligence method uses a two-layer feed-forward pattern-recognition neural network model in the Matlab toolbox. A total of 200 entropy values out of the five entropy matrixes for five different EEG channels was picked as the inputs of the neural network model, and four different sleep stages were defined as slow-wave sleep (SWS, including NREM3 and NREM4), light sleep (NREM1 and NREM2), REM, and wake stages for the training target of model. The performance of automatic sleep staging can be presented in the confusion matrix as shown in Table 2. The corrective percentages for four classes are 88.6, 85.8, 84.2, and 81.8% as the diagonal elements in the confusion matrix respectively. The consistency of the above four state classifications and target classifications are all greater than 80%. Therefore, the automatic sleep staging method provided by the present invention has a good accuracy rate, and the output results are highly matched with the manual scored sleep states.

TABLE 2

| | Target class | | | |
|---|---|---|---|---|
| Output class | Slow wave sleep | Light sleep | REM | Wake |
| Slow wave sleep | 88.6% | 6.0% | 0.4% | 0.9% |
| Light sleep | 11.1% | 85.8% | 14.5% | 6.9% |
| REM | 2.3% | 7.4% | 84.2% | 10.4% |
| Wake | 0.0% | 0.9% | 0.9% | 81.8% |

The above are only the preferred embodiments of the present invention, and do not limit the present invention in any form. Although the present invention has been disclosed as above in preferred embodiments, it is not intended to limit the present invention. Anyone who is familiar with the field, without departing from the scope of the technical solution of the present invention, can use the technical content disclosed above to make slight changes or modifications into equivalent embodiments with equivalent changes. Any simple modifications, equivalent changes and variations made to the above embodiments based on the technical essence of the present invention without departing from the technical solution of the present invention still fall within the scope of the technical solution of the present invention.

What is claimed is:

1. A construction method for automatic sleep staging, wherein it comprises the following steps:
acquiring a plurality of sets of polysomnography (PSG) signals and manual sleep information of PSG signals;
pre-analyzing to decompose the original time series of each stage in the PSG signals into a set of intrinsic mode functions (IMFs) or pseudo-intrinsic mode functions (pseudo-IMFs);
assembling the IMFs or pseudo-IMFs to obtain m sets of time series;
analyzing by multiscale entropy (MSE), to calculate the entropy values of the in sets of time series on n coarse-graining timescales, thus obtaining an entropy matrix with m*n elements;
defining the level of consciousness according to the manual sleep information;
establishing a correlation coefficient matrix between the level of consciousness and the elements in the entropy matrix, and finding the coarse-graining timescale and filtering timescale corresponding to the most significantly positively correlated element or the most significantly negatively correlated element in the correlation coefficient matrix, wherein the sampling timescale is coarse-graining timescale; and
calculating the entropy value on the coarse-graining timescale and filtering timescale corresponding to the most significantly positively correlated element or the most significantly negatively correlated element, and assessing the sleep state according to the entropy value.

2. The construction method for automatic sleep staging of claim 1, wherein the original time series of each stage in the PSG signals is decomposed into a set of IMFs using the mode decomposition method, and the mode decomposition method is one of the following methods: an empirical mode decomposition method, an ensemble empirical mode decomposition method, and a conjugate adaptive dyadic masking empirical mode decomposition method.

3. The construction method for automatic sleep staging of claim 1, wherein the original time series of each stage in the PSG signals is decomposed into a set of pseudo-IMFs using a set of high-pass filters, and the cut-off frequencies of the high-pass filters are 32 Hz, 16 Hz, 8 Hz, 4 Hz, 2 Hz, and 1 Hz, respectively.

4. The construction method for automatic sleep staging of claim 1, wherein the PSG signals comprise at least one of the following Electroencephalogram (EEG) signals: Fp4-A1, F4-A1, C4-A1, P4-A1, and O2-A1.

5. The construction method for automatic sleep staging of claim 1, wherein the level of consciousness is defined according to the manual sleep information, and the level of consciousness is used to reflect the degree of wakefulness during sleep, wherein a wake stage is quantified as 6, a rapid eye movement (REM) stage is quantified as 5, a non-rapid eye movement 1 (NREM1) stage is quantified as 4, an NREM2 stage is quantified as 3, an NREM3 stage is quantified as 2, and an NREM4 stage is quantified as 1.

6. The construction method for automatic sleep staging of claim 1, wherein the correlation coefficient matrix between the level of consciousness and the elements in the entropy matrix is established based on Pearson coefficient.

7. The construction method for automatic sleep staging of claim 1, wherein when assessing the sleep state according to the entropy value on the coarse-graining timescale and filtering timescale corresponding to the most significantly positively correlated element or the most significantly negatively correlated element, the threshold between different sleep states is calculated using the artificial intelligence (AI) method.

8. A method for automatic sleep staging, wherein it is a use of the method of claim 1, and it comprises the following steps:
acquiring PSG signals of a subject;
decomposing the PSG signals of the subject into original time series of a plurality of stages;
decomposing the original time series of a stage into a set of IMFs or pseudo-IMFs;
calculating the entropy value of the subject on the coarse-graining timescale and filtering timescale corresponding to the most significantly positively correlated element or the most significantly negatively correlated element of claim 1; and
assessing the sleep state of the subject at the stage according to the entropy value.

9. The method for automatic sleep staging of claim 8, wherein the PSG signals comprise at least one of the following Electroencephalogram (EEG) signals: Fp4-A1, F4-A1, C4-A1, P4-A1, and O2-A1.

10. The method for automatic sleep staging of claim 8, wherein when decomposing the PSG signals of the subject into original time series of a plurality of stages, the time of each stage is 30 seconds.
